(19) United States
(12) Patent Application Publication
(10) Pub. No.: US 2004/0089302 A1
(43) Pub. Date: May 13, 2004

(54) Breathing apparatus

(76) Inventor: Nicholas John Foss, West Essex (GB)

Correspondence Address: NATH & ASSOCIATES, 1030 15th STREET, 6TH FLOOR, WASHINGTON, DC, 20005, US

(21) Appl. No.: 10/415,591

(22) PCT Filed: Nov. 2, 2001
(86) PCT No.: PCT/GB01/04872

(30) Foreign Application Priority Data
Nov. 2, 2000 (GB) 0026825.0

(51) Int. Cl. A61M 16/00; A62B 7/00

(52) U.S. Cl. 128/204.18; 128/205.24; 128/204.21

(57) ABSTRACT

A breathing apparatus includes: supply means for supplying a breathable gas at a fist super-atmospheric pressure, face-piece means which can be worn by a user, the face-piece means being connected to the supply means and including an exhalation valve and a positive pressure demand valve for establishing a second super-atmospheric pressure, lower than said first super-atmospheric pressure, within the face-piece means when worn by the user, and a flow control device for controlling the flow of gas from the supply means to the face-piece means, the flow control device having a first operating position in which the flow gas to the demand valve is restricted and a second operating position in which the flow of gas to the demand valve is substantially unrestricted, and the flow control device being biased towards its first operating position and being moved to its second operating position by the establishment of the second super-atmospheric pressure within the face-piece means.

### FIELD OF THE INVENTION

This invention relates to breathing apparatus, particularly, but not exclusively, to self-contained breathing apparatus which can be used to enable the wearer to escape from areas of irrespirable atmosphere. Such apparatus typically includes a face-piece such as a flexible air-tight hood enclosing the wearer's head and sealed around the wearer's neck, the hood being supplied with air by a regulating means from a pressurised reservoir carried by the wearer.

Breathing apparatus incorporating face-pieces such as flexible hoods are well known and have been mostly of the “constant flow” type, in which air is supplied to the hood at a substantially constant rate, typically at about 40 litres per minute, which is generally accepted as being adequate for average consumption. However, as respiration is cyclic, the instantaneous flow rate required by the wearer will continually vary from zero during exhalation to about 125 litres per minute at the peak of each inhalation. It will thus be seen that a constant flow of 40 litres per minute will not satisfy the wearer's requirements unless the incoming air can be stored during exhalation in a flexible reservoir of sufficient volume to enable the wearer to inhale from that volume without restriction. In practice, the hood itself normally acts as the required reservoir and is made sufficiently large for this purpose.

A typical hood of the constant flow type is shown in FIG. 1 of the accompanying drawings and comprises a flexible hood A, a transparent visor area B and a neck seal C of elasticated fabric. Air is supplied from a high-pressure source (not shown), typically a cylinder containing air at 200 bar pressure. A valve (not shown) at the cylinder controls the flow of air to a pressure reducer (again not shown), which supplies air at about 10 bar to a flow-regulating valve which supplies the hood A. Air from the flow-regulating valve enters the hood A through a flexible hose D and a connector E and is directed over the visor area B by a deflector F to reduce misting. Surplus air and exhaled air can pass to atmosphere through the space between the neck seal C and the wearer's neck during exhalation.

There are a number of significant limitations inherent in the constant flow apparatus described above. Because the hood A acts as a reservoir into which the wearer exhales and from which he subsequently inhales, a significant proportion of the exhaled carbon dioxide will be re-inhaled. The effect of this is to stimulate more rapid breathing in an attempt by the wearer to reduce the carbon dioxide level in his or her lungs. As the rate of carbon dioxide production varies in dependence upon the wearer's physical condition and, most significantly, upon the rate at which he or she is working, it can be seen that, with a constant flow of fresh air into the hood 1, there will be a definite limit to the rate at which the wearer can work before the level of carbon dioxide within the hood A reaches a level high enough to cause distress. Thus, whilst the constant flow apparatus has the merits of simplicity and a predictable duration of flow from a given size of cylinder, these merits are, in practice, obtained at the expense of an adequate air supply to cater for the high demands that may be encountered in, for example, the stressful circumstances associated with escape from a contaminated area.

A further disadvantage of the constant flow hood apparatus is that the continuous inflation and deflation of the hood can cause aberration of the wearer's view through distortion of the visor. Also, the protection factor offered by the apparatus is poor, due to the fact that, during inhalation, the pressure within the hood may fall below that of the surrounding atmosphere with the result that, unless the integrity of the hood and its seal to the wearer's neck is very good, there may be some inward leakage of the surrounding atmosphere into the hood.

The deficiencies described above which are inherent in the constant flow hood apparatus can, to some extent, be overcome by supplying air on demand, rather than at a constant rate. In such a system, air is supplied as before from a high-pressure source, typically a cylinder containing air at 200 bar pressure. A valve at the cylinder controls the flow of air to a pressure reducer, which supplies air at about 10 bar to a positive pressure demand valve which regulates the flow of air precisely in accordance with the wearer's instantaneous requirements and, in conjunction with a spring-loaded exhalation valve, maintains a constant super-ambient pressure within the hood, effectively preventing any inward leakage.

A typical demand valve will have a sensitive pressure-responsive diaphragm, one face of which is exposed to the pressure within the hood and the other face exposed to ambient pressure. Movement of the diaphragm, in response to changes in the pressure within the hood, operates a valve to control the flow of air into the hood. The diaphragm is so biased as to close the valve when the pressure within the hood is, for example, 2 millibars above the ambient pressure. The exhalation valve, which allows the escape of surplus air to the atmosphere, is so biased as to open when the pressure within the hood is, for example, 3 millibars above the ambient pressure.

Thus, when the hood is sealed around the wearer's neck, a super-ambient pressure of between 2 and 3 millibars is maintained within the hood and the demand valve responds to the pressure changes caused by respiration and the admission of air into the hood in accordance with the user's requirements. The hood is kept constantly in an inflated condition, so that the visor remains in a substantially fixed position relative to the wearer's eyes and there is no flexing of the visor to distort the wearer's view.

Known hoods as described above still have significant disadvantages. Thus, in order to obtain a completely air-tight seal around the wearer's neck, a diaphragm seal is used. This consists of a disc of thin elastic material, such as latex, with a central hole for the neck, and can be difficult to put over the head, particularly by someone wearing spectacles, and can be prone to deterioration and tearing. The large volume of the hood necessitates the incorporation of an inner mask, covering the wearer's nose and mouth, to reduce the volume of the breathing circuit and so maintain an acceptably low level of inhaled carbon dioxide. It is necessary to secure this inner mask to the wearer's face in the correct position by means of an elastic or adjustable harness arrangement.

The edges of the inner mask, which project towards the wearer's face, are prone to catch on spectacles and thereby dislodge the spectacles, which can then be difficult to reposition within the hood. In addition, the inner mask can itself be pushed out of position when donning the hood, so that the inner mask has to be repositioned and secured to the wearer's face, in some cases by making adjustments to external straps. These are disadvantages which can adversely affect the ease and speed of donning the hood, these factors being of critical importance in emergency escape situations, particularly when the wearer has had limited training or experience in the use of the apparatus.

A further disadvantage of the known apparatus is that, in order to avoid a significant loss of air through the demand valve whilst the hood is being donned, it is necessary either to don the hood before opening the air supply from the cylinder or to provide the demand valve with what is known as a “first breath” mechanism, which prevents any flow of air into the hood until it is sealed to the wearer's neck and a partial vacuum can be drawn by the wearer's inhalation in order to operate the mechanism. Either of these arrangements causes further difficulty and delay in making the apparatus operational and an inexperienced user may be reluctant to don the hood if there is no apparent air supply to the hood.

A typical hood of the positive pressure type is shown in FIG. 2 of the accompanying drawings, in which like reference letters are applied to parts corresponding to those of FIG. 1. A flexible hood A has a transparent visor area B and is sealed around a wearer's neck by a neck seal C. A demand valve E is connected by a detachable coupling to an inner mask F via an adapter G. The inner mask F is held to the wearer's face by means of an elastic or adjustable harness H. Exhaled air escapes to atmosphere through a spring-loaded exhalation valve J rather than between the neck seal C and the wearer's neck.

It is an object of the present invention to provide an improved form of breathing apparatus.

In British Patent Specification No. 2 074 455, there is described a breathing apparatus which is so constructed that, if it is allowed to open fully, i.e. when no super-ambient pressure exists within the face-piece, the valve will close off the incoming air supply altogether. Thus, there will be no flow of air to the wearer at all while the face-piece is being donned and adjusted.

In U.S. Pat. No. 4,345,592, there is described a breathing apparatus which includes a device to completely close of the supply of air to the demand valve if the flow through the demand valve exceeds a predetermined rate, such as would be the case if the air supply were turned on prior to donning the face-piece or if the face-piece were removed with the air supply still turned on.

In U.S. Pat. No. 4,250,876, there is described a breathing apparatus which includes a demand valve provided with a two-position manually operated switch to either apply or remove a spring bias from the valve diaphragm such that, in one position, the demand valve functions in a positive pressure mode and, in the other position, the valve will operate in a negative pressure mode.

It is a more specific object of the present invention to provide a breathing apparatus which offers significant advantages as compared with the breathing apparatus described in the above specifications.

### SUMMARY OF THE INVENTION

According to the present invention there is provided a breathing apparatus which includes:

supply means for supplying a breathable gas at a first super-atmospheric pressure,

face-piece means which can be worn by a user, the face-piece means being connected to the supply means and including an exhalation valve and a positive pressure demand valve for establishing a second super-atmospheric pressure, lower than said first super-atmospheric pressure, within the face-piece means when worn by the user, and

a flow control device for controlling the flow of gas from the supply means to the face-piece means,

the flow control device having a first operating position in which the flow of gas to the demand valve is restricted and a second operating position in which the flow of gas to the demand valve is substantially unrestricted, and

the flow control device being biased towards its first operating position and being moved to its second operating position by the establishment of the second super-atmospheric pressure within the face-piece means.

The face-piece means may be a hood, a full face-piece helmet or a mouth-piece having a periphery sealable around a wearer's mouth.

Other preferred features of the invention are set out in the subsidiary claims.

### BRIEF DESCRIPTION OF THE DRAWINGS

FIG. 1, which has been referred to above, is a schematic side view of a constant flow hood,

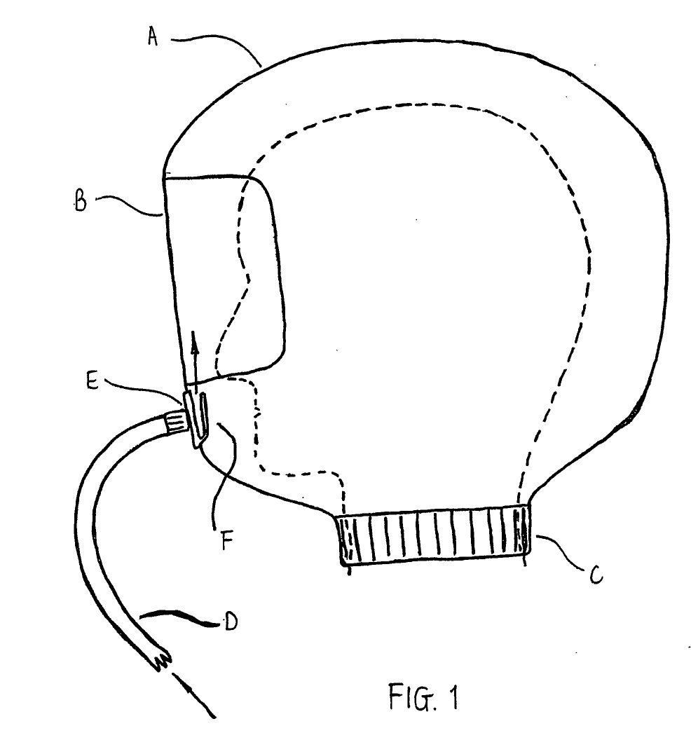

FIG. 2, which has also been referred to above, is a schematic side view of a positive-pressure type hood,

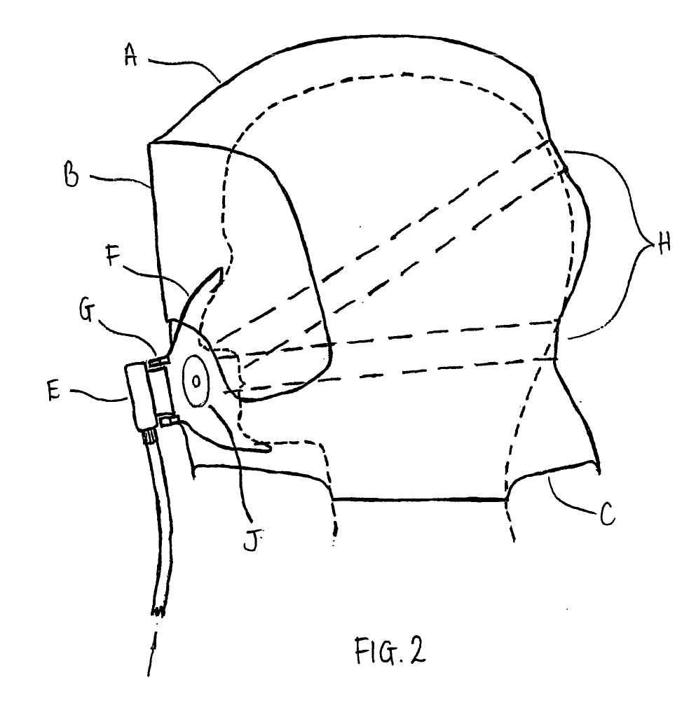

FIG. 3 is a schematic view of a breathing apparatus in accordance with the present invention incorporating a positive-pressure demand valve and a first form of air flow control valve,

FIG. 4 is a sectional view of the air flow control valve of FIG. 3 in a first operating position,

FIG. 5 is a view similar to FIG. 4, but showing the air control valve of FIG. 3 in a second operating position,

FIG. 6 is a sectional view of a second form of air flow control valve,

FIG. 7 shows a combined pressure-reducing valve and air flow control device when in an unpressurised state,

FIG. 8 shows the valve of FIG. 7 when connected to a pressurised reservoir,

FIG. 9 shows the valve of FIG. 7 in its restricted flow mode, and

FIG. 10 shows the valve of FIG. 7 in its full flow mode.

### DESCRIPTION OF THE PREFERRED EMBODIMENTS

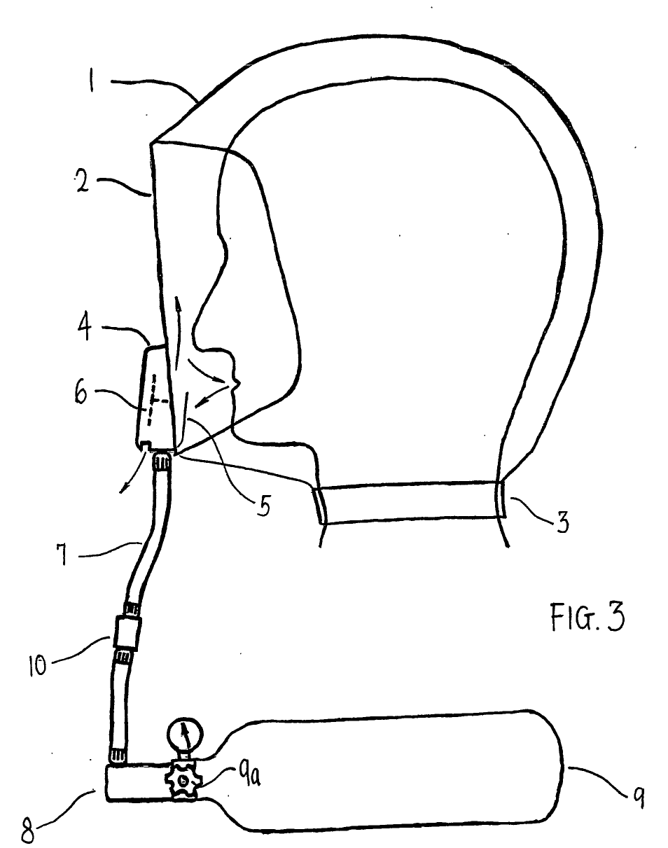

The breathing apparatus shown in FIG. 3 includes a hood 1 of flexible and impervious material and a clear visor area 2. The hood 1 is gathered around the neck where there is attached a neck seal 3 of fabric-reinforced elastic material, which can make an air-tight seal with a wearer's neck. A positive pressure demand valve 4 is incorporated into the construction of the hood 1 and has a deflector to guide incoming air, i.e. air entering the hood 1 from the valve 4, over the visor area 2 and so reduce misting. A spring-loaded exhalation valve 6 maintains a super-ambient pressure within the hood 1 and allows the escape of surplus air to atmosphere. Air is supplied to the demand valve 4 at a substantially constant pressure via a flexible hose 7 from a pressure-regulating valve 8 attached to a high pressure air reservoir or cylinder 9. A manually operated stop valve 9a controls the egress of air from the cylinder 9.

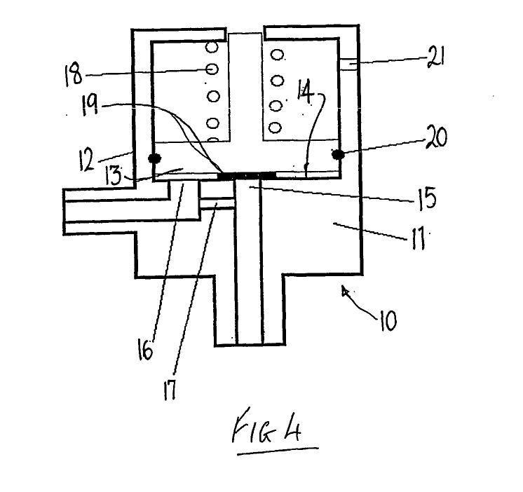

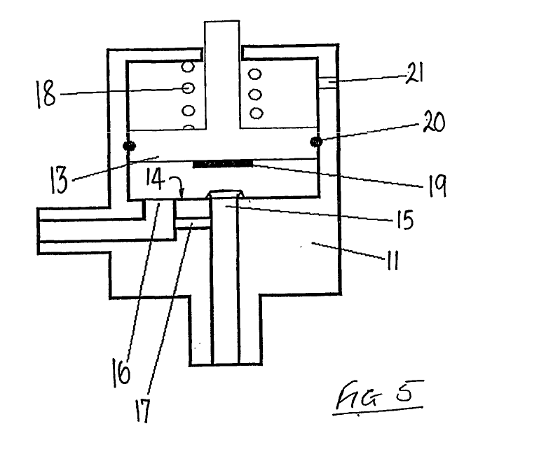

An air flow control device 10 is situated between the pressure-regulating (pressure-reducing) valve 8 and the demand valve 4. The air flow control device 10 has a first operating position, shown in FIG. 4, in which it restricts the flow of air into the hood 1 through the demand valve 4 to approximately 35 litres per minute, thus preventing significant loss of air whilst the hood 1 is being donned. When the hood 1 has been sealed around the wearer's neck, the pressure within the hood 1 will rise, thus closing the demand valve 4. This, in turn, will cause a rise in pressure at the inlet to the demand valve 4, and this rise in pressure will be sensed by the air flow control device 10, which will then adopt its second operating position, as shown in FIG. 5, in which the supply of air or the demand valve 4 is substantially unimpeded, the air flow control device 10 remaining in this second position throughout use

The first form of air flow control device 10 shown in FIGS. 4 and 5 comprises a housing 11 defining a cylinder 12 within which a piston 13 is movable. At one end face 14 of the cylinder 12, an inlet port 15 opens axially into the cylinder 12, and an outlet port 16 is provided at a position spaced radially outwardly from the inlet port 15. A transfer orifice 17 provides a restricted flow communication from the inlet port 15 to the outlet port 16. The piston 13 is urged towards the end face 14 of the cylinder 12 by a spring 18. The piston 13 carries a first sealing element 19 which is adapted to seal the inlet port 15 when the piston 13 is pressed against the end face 14 of the cylinder 12 by the spring 18. A second sealing element 20 extends around the periphery of the piston 13 to form a seal with the wall of the cylinder 12. The cylinder 12 is provided with a vent opening 21 in fluid connection with the ambient atmosphere, so that the face of the piston 13 remote from the end face 14 of the cylinder 12 is exposed to the ambient pressure.

In operation, the air flow control device 10 is connected between the pressure-regulating valve 8 and the demand valve 4, as shown in FIG. 3. When the apparatus is not in use, the valve 9a is closed and no pressurised air is applied to the air flow control device 10. The piston 13 is urged by the spring 18 towards the end face 14 of the cylinder 12 and the first sealing element 19 seals the inlet port 15.

When the apparatus is to be used, the user first opens the valve 9a so that compressed air is supplied from the cylinder 9 to the pressure-regulating valve 8 and thence to the air flow control device 10. Compressed air at a substantially constant pressure of about 10 bar enters the air flow control device 10 at the inlet port 15 which is effectively closed off by the first sealing element 19. The spring 18 applies a force to the piston 13 which is greater than the force applied by the air pressure acting on the area of the inlet port 15 and thus holds the piston 13 in the position shown in FIG. 4. The transfer orifice 17 allows a controlled continuous flow of air through the air flow control device 10 to the demand valve 4 and, as the demand valve 4 is normally open, this allows the air to flow freely into the hood 1. The pressure at the outlet port 16 of the air flow control device 10 is thus substantially the same as the ambient pressure so that the area of the piston 13 surrounding the inlet port 15 is thus exposed only to the ambient pressure and the piston 13 remains in its first operating position, as illustrated in FIG. 4. The user then dons the hood 1, and adjusts the neck seal 3 so as to provide an air-tight seal. The flow of air into the hood 1 reassures an inexperienced user of the reliability of the apparatus.

When the hood 1 is sealed around the wearer's neck, the incoming flow of air to the hood 1 through the transfer orifice causes the pressure within the hood 1 to rise. The rise in pressure is sensed by the demand valve 4 and eventually acts on the diaphragm of the demand valve 4 to close the demand valve 4, thus effectively closing off the outlet port 16 of the air flow control device 10. In consequence, the continuous flow of air through the transfer orifice 17 to the outlet port 16 causes the pressure in the ducting between the air flow control device 10 and the demand valve 4 to rise to the same pressure as that obtaining at the inlet port 15. This pressure acts, via the outlet port 16, upon the area of the piston 13 surrounding the inlet port 15 so that the entire face of the piston 13 which is adjacent to the end wall of the cylinder 12 is exposed to the inlet port pressure of approximately 10 bar. This produces a force on the piston 13 sufficient to overcome the opposing force applied by the spring 18 and the piston 13 is moved by this force away from the inlet port 15. The piston 13 is held continuously away from the inlet port 15 for as long as sufficient pressure is applied to the inlet port 15.

This movement of the piston 13 to the second operating position allows substantially unrestricted flow through the flow control device as and when the demand valve 4 opens in response to the users requirements. The piston 13 will remain in its second operating position until either the air supply is disconnected by the user closing the valve 9a or the air in the cylinder 9 is exhausted and the pressure at the inlet port 15 falls below a predetermined level. The force of the spring 18 will then overcome the force exerted on the face of the piston 13 by the inlet pressure and the piston 13 will return to the first operating position and seal the inlet port 15.

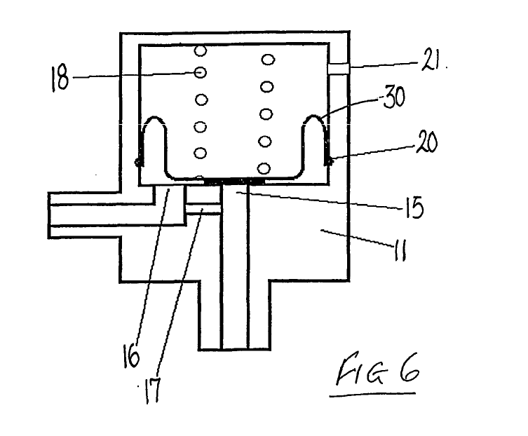

The construction of the air flow control device may differ from the specific arrangement shown in FIGS. 4 and 5. For example, in the air flow control device shown in FIG. 6, the piston 13 is replaced by a flexible diaphragm 30, the surface of which acts as the first sealing means. Alternatively, the inlet port 15 may be closed by a separate valve member operated by a piston or diaphragm. In a further alternative arrangement, the transfer orifice 17 may be constituted by a discontinuity in the sealing surfaces which close the inlet port so as to produce a controlled amount of leakage when the piston 13 is in its first operating position.

The air flow control device 10, although described in relation to the preceding embodiments as an independent assembly situated between the pressure-regulating valve 8 and the demand valve 4 may, in practice, be advantageously incorporated into the construction of the pressure regulator 8 or the demand valve 4, the latter design being of advantage when the breathing apparatus is supplied by a long hose from a remote source of compressed air.

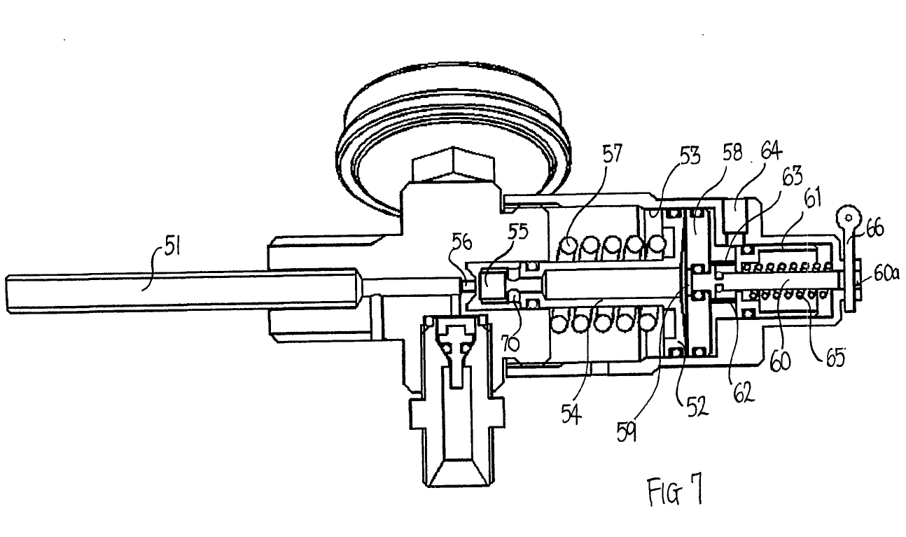

FIGS. 7 to 10 illustrate a combined pressure-reducing valve and air flow control device which comprises a valve body 50 having an inlet 51 connectable to a reservoir of high-pressure gas, such as a compressed air cylinder. A typical pressure at the inlet 51 may be 200 bar but pressures of up to 300 bar are possible.

The pressure-reducing valve comprises a piston 52 movable within a cylindrical bore 53 of the housing 50. The piston 52 is connected to one end of a hollow stem 54, the other end of which carries a sealing element 55 which co-operates with a high-pressure inlet port 56 supplied by the inlet 51. In the arrangement shown in FIGS. 7 to 10, a spring 57 urges the piston 52 to the right (as viewed in the drawings), so that the sealing element 55 is urged away from the inlet port 56.

The end of the cylindrical bore 53 is closed by a second piston 58 having a central through-bore 59. The through-bore 59 contains an O-ring which seals on one end of a control rod 60. The other end of the control rod 60 is slidingly received in a bore 60a in the housing 50. A third piston 61 has a central opening and an annular skirt 62 which surrounds the central opening and extends towards the second piston 58, the end surface of the annular skirt 62 forming a seal with the second piston 58. The central opening in the third piston 61 slidably engages the control rod 60 adjacent its one end, and is engageable with an O-ring seal provided on the control rod 60. The annular skirt 62 is formed with a bleed orifice 63, providing restricted fluid communication between the interior of the annular skirt 62 and an outlet port 64 in the housing 50. A spring 65 urges the third piston 61 towards the second piston 58. At the extreme right-hand end of the pressure-reducing valve (as viewed in FIGS. 7 to 10) there is a locking pin 66 which extends through a transverse bore intersecting the bore 60a. The locking pin 66 limits the extent of movement of the control rod 60 to the right.

The combined pressure-reducing valve and air flow control device shown in FIGS. 7 to 10 has four operating positions, the first of which is shown in FIG. 7. This corresponds to the state in which no pressure is present at the inlet 51. The spring 57 urges the piston 52 to the right, drawing the sealing element 55 away from the inlet port 56. The third piston 61 is urged to the left by the spring 65, so that the annular skirt 62 contacts the second piston 58 and, in turn, urges it to the left so as to contact the piston 52. The control rod 60, which is frictionally engaged by the O-ring seal in the second piston 58, is drawn to the left, away from the locking pin 66.

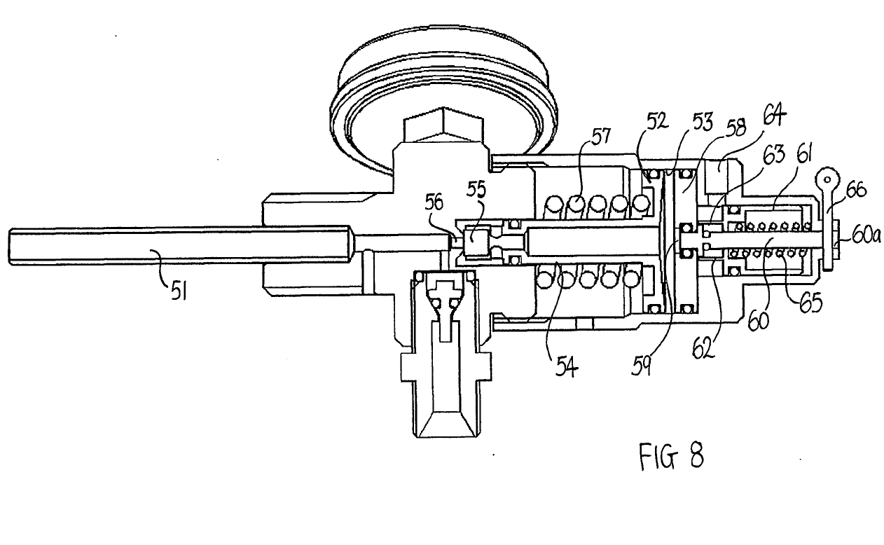

FIG. 8 shows the positions of the valve components when high pressure is present at the inlet 51. The high-pressure gas enters the inlet 51, and passes through inlet port 56, where its pressure is reduced to an intermediate pressure of, for example, 10 bar. A pair of transverse bores 70 admit the intermediate pressure gas to the centre of the hollow stem 54, and the space between the piston 52 and the second piston 58 is then raised to the intermediate pressure. This causes the second piston 58 to be moved to the right, until it contacts the end of the bore 53. Simultaneously, as the pressure in the space between the pistons 52 and 58 increases, the force on piston 52 overcomes the force of the spring 57, and piston 52 moves to the left. The sealing element 55 is thus moved towards the inlet port 56 and eventually closes the inlet port 56. In this position, the control rod 60 experiences the intermediate pressure on its left-hand end and is thus moved to the right, into contact with the locking pin 66. The movement of the piston 58 also moves the piston 61 to the right, slightly compressing the spring 65 and ensuring effective sealing contact between the annular skirt 62 and the piston 58.

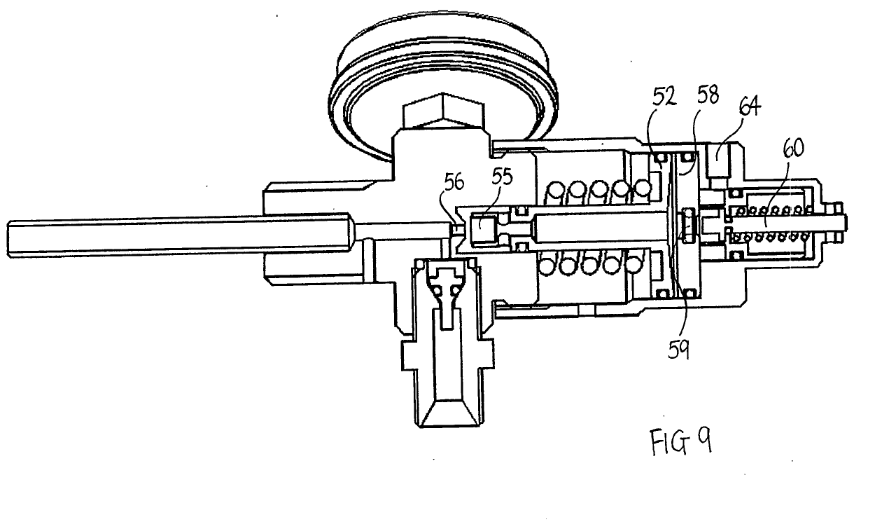

When it is required to provide breathable gas to a hood or other face-piece, the pin 66 is removed and the control rod 60 is then free to move to the right until its movement is arrested by a flange 60b contacting the face of the piston 61 between the annular skirt 62 and the central opening. In this position, which is shown in FIG. 9, the O-ring on the control rod 60 seals the central opening in the piston 61. This movement opens the central bore 59 in the piston 58, admitting fluid to the interior of the annular skirt 62. Simultaneously, the pressure in the space between the pistons 52 and 58 decreases and the spring 57 moves the piston 52 to the right, opening the inlet port 56 to admit more high-pressure gas. The bleed orifice 63 in the skirt 62 allows a restricted flow of gas to the outlet port 64, and thence to a demand valve of the breathing apparatus. The demand valve will be in an open condition, and thus the pressure sensed at the outlet port 64 will be substantially atmospheric pressure. The size of the bleed orifice 63 is so chosen that it provides the required volumetric flow rate when the pressure loss across the bleed orifice 63 corresponds to the difference between the intermediate pressure and atmospheric pressure.

The intermediate pressure acting within the annular skirt 62 produces insufficient force to overcome the force exerted by the spring 65, assisted by atmospheric pressure, acting on the right-hand face of the piston 61 so that the annular skirt 62 of the piston 61 is kept in sealing contact with the second piston 58. Thus, in the initial flow state after removal of the locking pin 66, the space between the pistons 52 and 58 is provided with gas at an intermediate pressure of, for example, 10 bar, while the flow control valve components 58, 60 and 61 provide a regulated volumetric flow rate to the outlet port 64 via the central bore 59 and the bleed orifice 63.

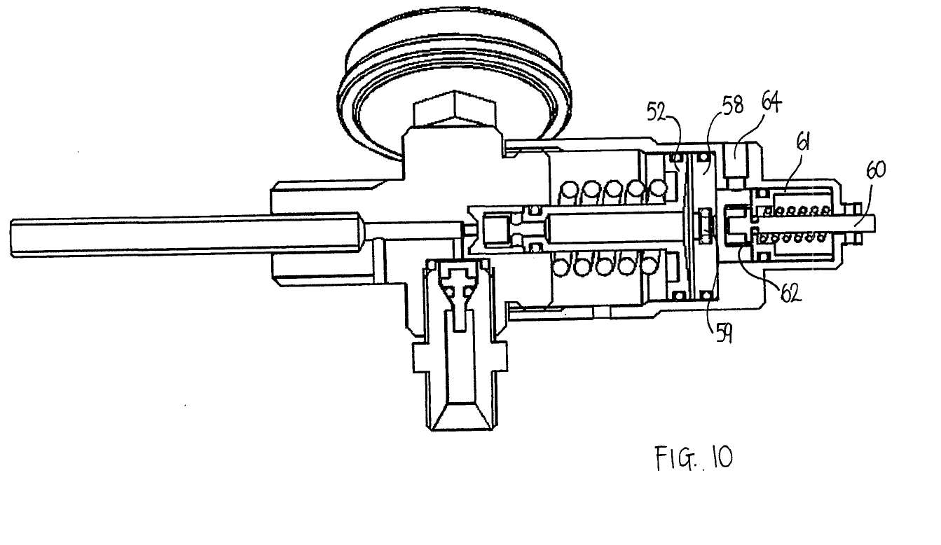

FIG. 10 shows the positions of the valve components when a back pressure is sensed at the outlet port 64, for example, when a wearer dons a face-piece or hood having a demand valve to which the outlet port 64 supplies breathable gas. In this condition, when the demand valve closes due to pressure within the hood or face-piece, the pressure in the space between the piston 58 and the area of the piston 61 outside the annular skirt 62 increases, and eventually equalises with the intermediate pressure within the annular skirt 62. The stiffness of spring 65 is so chosen that, when the entire leftward-facing area of the piston 61 is exposed to this intermediate pressure, the force on the piston 61 moves the piston 61 to the right, until the piston 61 contacts the end surface of its associated bore.

This movement of the piston 61 separates the annular skirt 62 from the face of the piston 58, and thus provides a substantially unrestricted flow path for gas to flow to the outlet port 64 at the intermediate pressure established by the pressure-reducing valve components 52, 55 and 56. When the demand valve opens to admit gas to a face-piece or hood, the pressure acting on the right-hand face of the piston 52 reduces, and thus the spring 57 moves the sealing element 55 away from the inlet port 56 to increase the rate of flow of gas therethrough. Likewise, when the demand valve closes, the pressure acting on the piston 52 increases and the sealing element 55 is urged towards the inlet port 56 to reduce or arrest the flow of gas.

To increase the usefulness of the combined pressure-reducing valve and air flow control device shown in FIGS. 7 to 10, a charging port 70 is provided. The charging port 70 can be attached to a high-pressure source of gas in order to replenish a cylinder attached to the inlet 51. Replenishment will take place with the pin 66 in position, and with the valve components in their positions shown in FIG. 8. When gas pressure at the charging port 70 exceeds the gas pressure obtaining in a reservoir connected to the inlet 51, a non-return valve 71 will be lifted and gas will flow from the charging port 70 into the reservoir. Disconnection of the supply from the charging port 70 causes the non-return valve 71 to close, preventing leakage of gas from the reservoir.

As a further safety feature, the combined pressure-reducing valve and air flow control device shown in FIGS. 7 to 10 may be provided with an over-pressure relief arrangement such as a bursting disc or other pressure-limiting device, in fluid communication with the inlet 51.

It is envisaged that the combined pressure-reducing valve and air flow control device shown in FIGS. 7 to 10 may be provided in association with a hood or other face-piece and a reservoir of breathable gas in an “escape set”, preferably packaged in a protective container for emergency evacuation of personnel from a building or vessel. The container may be a flexible protective fabric bag, or a substantially rigid casing. The container may be attached to a wall of the building or vessel.

In an advantageous form of “escape set”, the pin 66 may be attached by a lanyard to the container so that, when the escape set is removed from the container, the pin 66 is withdrawn and the restricted flow of gas to the face-piece is automatically established without the user having to operate any valve manually. Thus, while the user is donning the hood or face-piece, only a limited flow of gas is permitted and the reservoir is prevented from becoming prematurely depleted. Once the user has donned the hood or face-piece, operation of the demand valve will cause the flow control valve elements 58, 60 and 61 to assume the positions shown in FIG. 10, allowing the full flow of gas to the user. The restricted gas flow provided while the user is donning the hood or face-piece provides reassurance to the user that, when he or she has the hood or face-piece fully in place, a supply of breathable gas will become available.

It will be appreciated that the present invention is not limited to breathing apparatus for escape purposes, but may be applied with equal advantage to breathing apparatus for other purposes and utilising other forms of hood, face-piece or helmet, or a mouth-piece having a flexible seal extending around its periphery for sealing engagement around the mouth of a wearer of the face-piece. For example, the breathing apparatus may be provided for use in hazardous industrial environments such as paint spray shops, in which each worker has a hood or face-piece provided with a breathable gas through a compressed air hose from a central source of supply. The connection to the supply hose is generally made with a coupling which closes the supply hose when disconnected, to prevent loss of gas. The workers can thus connect their hoods or face-pieces to the supply and don the hood or face-piece while a restricted flow of gas is supplied via the control valve. The wearer than has confidence in the apparatus, and minimum loss of breathable gas occurs during this phase of operation.

1. A breathing apparatus which includes:—
supply means for supplying a breathable gas at a first super-atmospheric pressure,
face-piece means which can be worn by a user, the face-piece means being connected to the supply means and including an exhalation valve and a positive pressure demand valve for establishing a second super-atmospheric pressure, lower than said first super-atmospheric pressure, within the face-piece means when worn by the user, and
a flow control device for controlling the flow of gas from the supply means to the face-piece means,
the flow control device having a first operating position in which the flow of gas to the demand valve is restricted and a second operating position in which the flow of gas to the demand valve is substantially unrestricted, and
the flow control device being biased towards its first operating position and being moved to its second operating position by the establishment of the second super-atmospheric pressure within the face-piece means.

2. A breathing apparatus as claimed in claim 1, in which the flow control device includes:—
an inlet port,
an outlet port,
sealing means movable between a first position in which the sealing means closes the inlet port and a second position in which the inlet port is open, the first and second positions of the sealing means corresponding to the first and second operating positions respectively of the flow control device,
a by-pass providing restricted fluid communication between the inlet and outlet ports irrespective of the position of the sealing means,
biasing means to urge the sealing means towards its first position, and
actuating means responsive to fluid pressure at the outlet port and operable to move the sealing means from the first position to the second position when the fluid pressure at the outlet port exceeds a predetermined threshold value.

3. A breathing apparatus as claimed in claim 2, in which the actuating means includes a movable surface urged in a first direction by the biasing means and urged in a second direction, opposite to the first direction, by having a first region exposed to the fluid pressure at the inlet port and a second region exposed to the fluid pressure at the outlet port.

4. A breathing apparatus as claimed in claim 2 or claim 3, in which the actuating means includes a movable piston.

5. A breathing apparatus as claimed in claim 2 or claim 3, in which the actuating means includes a flexible diaphragm.

6. A breathing apparatus as claimed in any one of claims 2 to 5, in which the by-pass is defined between the inlet port and the sealing means when the sealing means is in its first position.

7. A breathing apparatus as claimed in any one of claims 2 to 5, in which the by-pass includes a passage extending from the inlet port to the outlet port independently of the sealing means.

8. A breathing apparatus as claimed in any one of claims 2 to 5, in which the by-pass includes a passage extending through the sealing means.

9. A breathing apparatus as claimed in any one of claims 2 to 8, in which the flow control device includes releasable retaining means for retaining the sealing means in its first position.

10. A breathing apparatus as claimed in claim 9, in which the releasable retaining means includes a removable locking element.

11. A breathing apparatus as claimed in any one of the preceding claims, in which a pressure-reducing valve is provided between the supply means and the flow control valve.

12. A breathing apparatus as claimed in claim 11, in which the flow control valve and the pressure-reducing valve are contained in a common housing.

13. A breathing apparatus as claimed in any one of claims 1 to 11, in which the flow control valve and the demand valve are contained in a common housing.

14. A breathing apparatus as claimed in any one of the preceding claims, in which the face-piece means includes a hood.

15. A breathing apparatus as claimed in any one of claims 1 to 13, in which the face-piece means includes a helmet.

16. A breathing apparatus as claimed in any one of claims 1 to 13, in which the face-piece means includes a mouth-piece having a periphery sealable around a wearer's mouth.

17. A breathing apparatus as claimed in any one of the preceding claims, in which the supply means includes a reservoir of gas at super-atmospheric pressure.

18. A breathing apparatus as claimed in any one of claims 1 to 16, in which the supply means includes a compressor for supplying gas at super-atmospheric pressure.
